(19) United States
(12) Patent Application Publication
(10) Pub. No.: US 2026/0166261 A1
(43) Pub. Date: Jun. 18, 2026

(54) 3D PRINTING OF CATHETER SHAFTS

(71) Applicant: MEDIBRANE LTD

(72) Inventors: Elad EINAV, San Jose, CA (US); Liliya KOPEL, Barkan (IL)

(21) Appl. No.: 19/416,698

(22) Filed: Dec. 11, 2025

Related U.S. Application Data
(63) Continuation of application No. PCT/IB2024/055849, filed on Jun. 14, 2024.
(60) Provisional application No. 63/472,881, filed on Jun. 14, 2023.
(60) Provisional application No. 63/536,942, filed on Sep. 7, 2023.
(60) Provisional application No. 63/567,668, filed on Mar. 20, 2024.

(51) Int. Cl. A61M 25/00; B33Y 80/00

(52) U.S. Cl. CPC A61M 25/0012; A61M 25/0045; A61M 25/005; B33Y 80/00; A61M 2207/00

(57) ABSTRACT

A catheter shaft comprises a liner layer surrounding an elongated lumen, a reinforcement layer at least partly surrounding the liner layer and arranged coaxially therewith, and a jacketing array at least partly surrounding the reinforcement layer and arranged coaxially therewith. The jacketing array comprises at least 10 polymeric jacket layers each having a minimum thickness of at least 0.5 microns and a maximum thickness of at most 10 microns. A system for manufacturing a catheter shaft comprises a plurality of spaced-apart holder assemblies configured to support an elongated mandrel therebetween, and one or more nozzles, arranged to move along a length of the mandrel and deposit, the liner layer and/or the jacket layers of the jacketing array.

### CROSS-REFERENCE TO RELATED APPLICATIONS

The present application is a continuation of PCT/IB2024/055849, filed on Jun. 14, 2025, which is incorporated herein by reference in its entirety.

### FIELD OF THE INVENTION

The present invention generally relates to medical apparatuses and their manufacture, and in particular to catheter shafts and polymeric tubes manufactured using 3D printing deposition techniques.

### BACKGROUND

Intravascular catheters and other diagnostic or therapeutic catheters for use within the body for treatment and diagnosis of diseases are generally known. Examples of catheters include guide catheters, angioplasty catheters, stent delivery devices, angiographic catheters, neuro catheters, and the like. Most of these catheters are intended to be left within the body for an extended period of time. Therefore, the catheter needs to be constructed to have better maneuverability, manipulation, and activation with minimum danger to the patient.

Most existing therapeutic catheters used in the field of brain surgery or the circulatory system are built in a way to require a lot of manual force, which makes the production costs high. As a result, many innovative treatments do not reach patients in need. Due to the relatively small quantities of catheters, it is difficult to streamline the process and justify the manufacturing investment to companies. Each catheter would require a separate dedicated production line and involves many manufacturing stages.

In conventional methods of manufacturing, various layers are produced using various techniques and then manually assembled, which increases the cost of manufacturing and is prone to errors.

Therefore, there is a need for cost-effective systems and methods that decrease the extent of required manual work and improves quality and repeatability.

### SUMMARY

According to the embodiments disclosed herein, a catheter shaft produced using polymer deposition comprises: (a) a liner layer surrounding an elongated lumen; (b) a reinforcing layer at least partly surrounding the liner layer and arranged coaxially therewith, the reinforcing layer comprising a metal or metal alloy; and (c) a jacketing array at least partly surrounding the reinforcing layer and arranged coaxially therewith, the jacketing array comprising a plurality of deposited polymeric jacket layers (e.g., at least 5 deposited polymeric jacket layers) each having a minimum thickness of at least 1 micron and a maximum thickness of at most 5 microns. At least one of the following is true: (i) the catheter shaft comprises first and second longitudinal sections characterized by different respective hardness values and separated by a transition section having a length of at least 5 mm and not more than 30 mm, the transition section being characterized by a gradation of hardness values between the different respective hardness values, and/or (ii) the catheter shaft comprises first and second longitudinal sections comprising different respective jacket-layer material compositions and separated by a transition section having a length of at least 5 mm and not more than 30 mm, the transition section comprising gradated intermediate blends of the different jacket-layer material formulations.

In some embodiments, catheter shaft can comprise first and second longitudinal sections separated by a transition section having a length of at least 5 mm and not more than 30 mm, the first and second longitudinal sections comprising different respective jacket-layer material compositions each characterized by a different respective hardness value, wherein the transition section comprises gradated intermediate blends of the different jacket-layer material formulations and is characterized by a gradation of hardness values between the different respective hardness values.

In some embodiments, the jacketing array can comprise at least 5 jacket layers each having a minimum thickness of at least 1 micron and a maximum thickness of at most 5 microns. In some embodiments, the jacketing array can comprise at least 10jacket layers each having a minimum thickness of at least 1 micron and a maximum thickness of at most 5 microns.

In some embodiments, the liner layer can have a higher hardness (durometer) value than at least one of the jacket layers. In some embodiments, the liner layer can have a higher hardness value than any of the jacket layers.

In some embodiments, it can be that a ratio of a maximum jacket-layer thickness to a diameter of the catheter shaft is not more than 0.2%.

In some embodiments, a lengthwise majority of the catheter shaft can be characterized by a monotonically increasing or decreasing inner diameter. In some embodiments, a lengthwise majority of the catheter shaft can be characterized by a monotonically increasing or decreasing outer diameter.

A method is disclosed, according to embodiments, for of manufacturing the catheter shaft of any one of the foregoing embodiments, the method comprising: (a) providing the inner layer and the reinforcement layer such that the inner layer is disposed around a portion of an elongated mandrel disposed between two holder assemblies and the reinforcement layer is disposed around at least a portion of the inner layer; and (b) sequentially depositing each of the jacket layers over the reinforcement layer to form the jacketing array.

In some embodiments, providing the inner layer can include forming the inner layer by depositing a polymeric coating over the mandrel.

In some embodiments, it can be that none of the jacket layers are formed by extrusion or injection molding.

In some embodiments, a system for manufacturing a catheter shaft according to any one of the foregoing embodiments can comprise: (a) a plurality of spaced-apart holder assemblies configured to support an elongated mandrel therebetween, at least one of the holder assemblies further configured to apply a tension to the mandrel; and (b) one or more nozzles, arranged to move along a length of the mandrel and to deposit thereupon (on the mandrel and/or on a previously deposited layer) a jacket layer of a polymeric jacketing array. In some embodiments, the system can additionally comprise a curing element arranged to move along the length of the mandrel and cure the deposited layer, the curing element comprising at least one of an air tube, a heater, an RF emitter, a UV light emitter, and an IR light emitter.

A method is disclosed, according to embodiments, for manufacturing a catheter shaft, The method comprises: (a) providing a liner layer, disposed around a mandrel extending between two holder assemblies; (b) providing a reinforcement layer over the liner layer; (c) sequentially depositing each of a plurality of polymeric jacket layers (e.g., at least 5 polymeric jacket layers) over the reinforcement layer to form a jacketing array, each jacket layer having a minimum thickness of at least 1 micron and a maximum thickness of at most 5 microns; and (d) removing the mandrel to create an elongated lumen surrounded by the liner layer.

In some embodiments, the sequentially depositing can additionally comprise curing the jacket layer, wherein the curing includes at least one of drying, heating, exposing to a selected radio frequency (RF), and irradiating with UV or IR light.

In some embodiments, providing the liner layer can include forming the liner layer by depositing a polymeric coating over the mandrel.

In some embodiments, the method can additionally comprise applying a surface treatment to the reinforcement layer.

In some embodiments, the sequentially depositing can comprise, for at least one of the jacket layers, forming first and second longitudinal sections characterized by different respective hardness values and a transition section therebetween having a length of at least 5 mm and not more than 30 mm, wherein the transition section can be characterized by a gradation of hardness values between the different respective hardness values. In some embodiments, the sequentially depositing can comprise, for at least one of the jacket layers, forming first and second longitudinal sections comprising different respective jacket-layer material compositions and separated by a transition section having a length of at least 5 mm and not more than 30 mm, wherein the transition section can comprise gradated intermediate blends of the different jacket-layer material formulations. In some embodiments, the sequentially depositing can comprise, for at least one of the jacket layers, forming first and second longitudinal sections separated by a transition section having a length of at least 5 mm and not more than 30 mm, the first and second longitudinal sections comprising different respective jacket-layer material compositions each characterized by a different respective hardness value, wherein the transition section can comprise gradated intermediate blends of the different jacket-layer material formulations and is characterized by a gradation of hardness values between the different respective hardness values.

Some embodiments disclose a catheter shaft produced using any of the foregoing methods.

According to the embodiments disclosed herein, a system for producing a catheter shaft comprises: (a) a plurality of spaced-apart holder assemblies configured to support an elongated mandrel therebetween, at least one of the holder assemblies further configured to apply a tension to the mandrel; and (b) one or more nozzles, arranged to move along a length of the mandrel and deposit a jacket layer of a jacketing array while moving, the nozzles configurable to form jacket layers each having a minimum thickness of at least 1 micron and a maximum thickness of at most 5 microns.

In some embodiments, the system can additionally comprise a curing element arranged to move along the length of the mandrel and cure the deposited layer while moving, the curing element comprising at least one of an air tube, a heater, an RF emitter, a UV light emitter, and an IR light emitter.

In some embodiments, it can be that an outer surface of the mandrel is characterized by an average surface roughness of 15 to 45 microns. In some embodiments, the mandrel can comprise one or more of: silver-coated copper, silver-coated aluminum, and PTFE reinforced with glass fibers. In some embodiments, it can be that the mandrel has a ductility of at least 25%.

In some embodiments, the system can additionally comprise a control system programmed to regulate the operation of the one or more nozzles to: (i) form a jacket layer comprising first and second longitudinal sections characterized by different respective hardness values and separated by a transition section having a length of at least 5 mm and not more than 30 mm, wherein the transition section can be characterized by a gradation of hardness values between the different respective hardness values, (ii) form a jacket layer comprising first and second longitudinal sections comprising different respective jacket-layer material compositions and separated by a transition section having a length of at least 5 mm and not more than 30 mm, wherein the transition section can comprise gradated intermediate blends of the different jacket-layer material formulations, and/or (iii) form a jacket layer comprising first and second longitudinal sections separated by a transition section having a length of at least 5 mm and not more than 30 mm, the first and second longitudinal sections comprising different respective jacket-layer material compositions each characterized by a different respective hardness value, wherein the transition section can comprise gradated intermediate blends of the different jacket-layer material formulations and is characterized by a gradation of hardness values between the different respective hardness values.

In some embodiments, the control system can additionally be programmed to regulate loading of the mandrel on or through the holding assemblies and/or to regulate cutting the mandrel after the producing. In some embodiments, the control system can additionally be programmed to regulate a thickness of a deposited and cured jacket layer.

According to embodiments disclosed herein, a polymer tube produced using polymer deposition comprises an array of polymeric layers surrounding an elongated lumen and comprising a plurality of deposited polymeric layers (e.g., at least 5 deposited polymeric layers) each having a minimum thickness of at least 1 micron and a maximum thickness of at most 5 microns. At least one of the following is true: (i) the polymer tube comprises first and second longitudinal sections characterized by different respective hardness values and separated by a transition section having a length of at least 5 mm and not more than 30 mm, the transition section being characterized by a gradation of hardness values between the different respective hardness values, and/or (ii) the polymer tube comprises first and second longitudinal sections comprising different respective polymeric-layer material compositions and separated by a transition section having a length of at least 5 mm and not more than 30 mm, the transition section comprising gradated intermediate blends of the different polymeric-layer material formulations.

In some embodiments, the polymer tube can comprises first and second longitudinal sections separated by a transition section having a length of at least 5 mm and not more than 30 mm, the first and second longitudinal sections comprising different respective polymeric-layer material compositions each characterized by a different respective hardness value, wherein the transition section can comprise gradated intermediate blends of the different polymeric-layer material formulations and is characterized by a gradation of hardness values between the different respective hardness values.

In some embodiments, a system for manufacturing the polymer tube of any of the foregoing embodiments can comprise: (a) a plurality of spaced-apart holder assemblies configured to support an elongated mandrel therebetween, at least one of the holder assemblies further configured to apply a tension to the mandrel; and (b) one or more nozzles, arranged to move along a length of the mandrel and to deposit thereupon (on the mandrel and/or on a previously deposited layer) a polymeric layer.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. One dimension can be exaggerated relative to another dimension.

Embodiments incorporating teachings of the present disclosure are shown and described with respect to the figures presented herein, in which:

FIGS. 1, 2, 3A, 3B, and 4 schematically show various perspective and cutaway views of various catheter shafts according to embodiments of the present invention.

FIG. 5 shows flowchart of a method of manufacturing a catheter, according to embodiments of the present invention.

FIG. 6 shows a schematic cutaway view of a catheter shaft according to embodiments of the present invention.

FIG. 7 is ae schematic illustration of a catheter shaft inside a surgical path, according to embodiments of the present invention.

FIGS. 8, 9 and 10 are schematic illustrations of exemplary catheter shafts that include binding polymer layers, according to embodiments of the present invention.

FIGS. 11 and 12 are schematic illustrations of a manufacturing setup for catheters according to embodiments of the present invention.

FIG. 13A is a schematic cross-section showing an arrangement of various layers of a catheter shaft, according to embodiments of the present invention.

FIGS. 13B and 13C show designs for a detail of FIG. 13A, according to embodiments of the present invention.

FIGS. 14A, 14B and 14C show designs for a detail of FIG. 13A including a transition section between two longitudinal sections characterized by different material properties, according to embodiments of the present invention.

FIGS. 15 and 16 are schematic illustrations of catheter shafts respectively having non-constant outer and inner diameters, according to embodiments of the present invention.

FIGS. 17A and 17B schematically illustrate an exemplary mandrel fixed between holder assemblies, respectively with and without slack along the length of the mandrel, according to embodiments of the present invention.

FIGS. 18, 19, 20, 21, 22, 23, 24, 25 and 26A schematically illustrate respective systems for manufacturing a catheter shaft, according to embodiments of the present invention.

FIG. 26B schematically illustrates a reinforcement layer being disposed over the liner layer, according to embodiments of the present invention.

FIG. 26C schematically illustrates a nozzle depositing drops of a polymer over a substrate to form a polymer layer, according to embodiments of the present invention.

FIGS. 27A and 27B schematically illustrate provision of a mandrel with attached tip and a handle as part of a system for manufacturing a catheter shaft, according to embodiments of the present invention.

FIG. 28 schematically illustrates use of a balloon-type mandrel in a system for manufacturing a catheter shaft, according to embodiments of the present invention.

FIG. 29 schematically illustrates use of a cone-type mandrel in a system for manufacturing a catheter shaft, according to embodiments of the present invention.

FIG. 30 schematically illustrates an exemplary tube integrated with nozzles, air tubes, solvent tubes and magnetic needle, according to embodiments of the present invention.

FIG. 31 is a schematic representation of layers of deposited drops of polymer, according to embodiments of the present invention.

FIGS. 32, 33A, 33B, 33C and 33D show flowcharts of respective methods and method steps for manufacturing a catheter shaft, according to embodiments of the present invention.

FIG. 34A is a schematic cross-section showing an arrangement of various layers of a polymeric tube, according to embodiments of the present invention.

FIG. 34B shows a detail of FIG. 34A, according to embodiments of the present invention.

FIGS. 35A, 35B and 35C show flowcharts of respective methods and method steps for manufacturing a polymeric tube, according to embodiments of the present invention.

### DETAILED DESCRIPTION OF EXAMPLE EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Throughout the drawings, like-referenced characters are generally used to designate like elements. Subscripted reference characters (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

Embodiments disclosed herein relate to systems and methods for manufacturing catheter shafts and polymeric tubes are disclosed herein. Catheter shafts and tubes are produced by various additive-manufacturing techniques including polymer deposition. Systems and methods for manufacturing the catheter shafts and polymeric tubes use arrays of one or more nozzles or, equivalently, other deposition devices such as, for example, plasma jets aligned along, e.g., above, one or more elongated mandrels arranged to be coated and surrounded by various deposited polymer layers.

In the instant disclosure, the term “deposition” is used to represent the broad range of additive production technologies disclosed. The term is used without limitation and in practice include any application of fine polymeric particles, e.g., entrained in solutions, suspensions and/or plasma streams. Application of fine polymeric particles can include dispersion of fine particles in any material phase and can include breaking down a substance into small droplets or particles so as facilitate achieving uniform coverage or distribution over a targeted area. The size, density, velocity, and/or dispersion patterns of the polymeric particles can depend, for example, on the application method and the properties of the substance being applied. Examples of technologies embodied in the term ‘deposition’ (or ‘depositing’, etc.) include, and not exhaustively, a spray process, droplet deposition, electrospinning, and electrospray technology. The terms ‘segment’ and ‘section’, when used in the context of longitudinal divisions of a catheter shaft, polymeric tube, or a polymeric layer, are synonymous with each other and are used interchangeably.

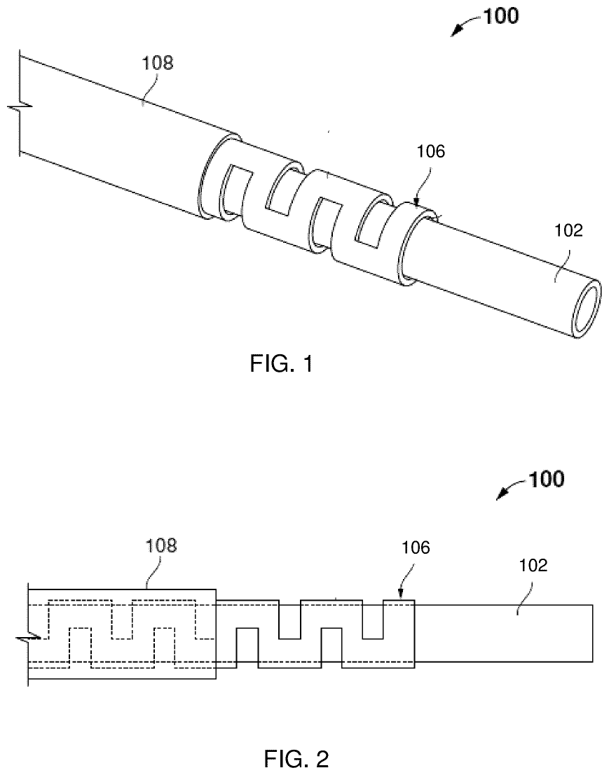

Referring to the figures, and in particular to FIGS. 1 and 2, a first nonlimiting example of a catheter shaft 100 is illustrated. Catheter shafts can be longer than 50 cm and can even be longer than 1 meter, while the diameter of a catheter shaft can be as small as 1 mm and generally less than 5 mm or 10 mm. In some embodiments, the catheter shaft 100 comprises a reinforcement layer 106, provided over an inner liner layer 102. The reinforcement layer 106 is shown in FIGS. 1 and 2 as a hypotube with an open-surface design, e.g., a hypotube laser-cut to open ‘windows’ in the elongated cylindrical tube, e.g., to increase flexibility and/or to allow mechanical communication through the walls of the reinforcement layer 106 between a jacketing array 108 and an inner polymeric liner layer 102. The liner may be from polyurethane (PTFE) or expanded PTFE (ePTFE) or silicone or any dissolvable material. In some embodiments, the liner may be designed from ePTFE with several layers with a plurality of pores in it. In some embodiments, the liner made from ePTFE is designed to expand longitudinally along the shaft axis. In some embodiments, the liner may include one or more sections with different hardness values—for examples, different hardness values far apart on the Shore hardness scales such as 40 A (±10 ) and 70 D (±10).

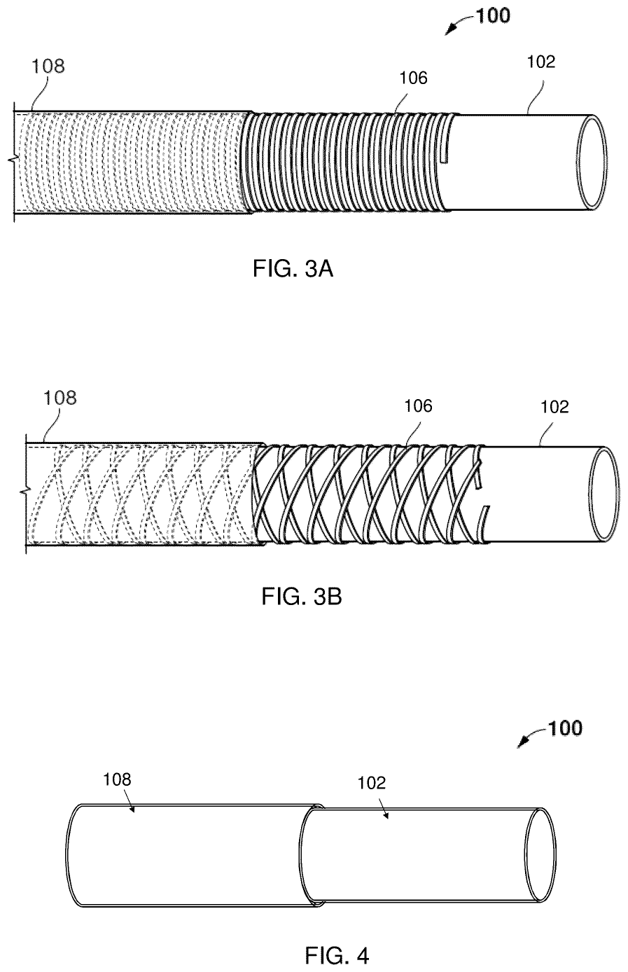

In some embodiments, the reinforcement layer 106 is a laser-cut hypotube having a diameter ranging from about 0.2 mm to about 10 mm, produced from various materials including metals and metal alloys such as, and not exhaustively, nitinol or stainless steel. Other non-limiting examples of reinforcement layers 106 include other open-structure designs such as a coil (as shown in FIG. 3A) or a braid (as shown in FIG. 3B).

In embodiments, the catheter shaft 100 further comprises a jacketing array 108 comprising a plurality of polymeric jacket layers deposited or printed onto a reinforcement layer 106. In embodiments, one or more (or all) of the jacket layers can have two different hardness values (‘durometer’ values), i.e., the hardness of the materials differs in different longitudinal sections of the catheter shaft 100. In some embodiments, a transition section between the two longitudinal sections bridges the two values. In embodiments, one or more (or all) of the jacket layers can comprise two different material compositions, i.e., in different longitudinal sections of the catheter shaft 100. In some embodiments, a transition section between the two longitudinal sections bridges the two hardness values and/or material composition, for example by having a gradated intermediate hardness and/or gradated blend of the two different material compositions. In some embodiments, the different hardness-value materials of the jacket layers are selected from the group comprising polyurethane, PEBA, and silicone. In some embodiments, the polymer jacketing array 108 is created on the reinforcement layer with at least 2 different wall thickness.

In some embodiments, the polymer jacketing array 108 is fixedly attached to the reinforcement layer 106 by creating a covalent connection between the metal surface of the reinforcement layer 106 to the polymer of the jacket layers. The reinforcement layer 106 can undergo surface treatment or be coated with a primer in order to create the covalent bonds

In some embodiments, the catheter shaft 100 is provided with the polymer jacketing array 108 and without a liner 102. In some embodiments, the jacketing array 108 is porous. In some embodiments, as illustrated in FIG. 4, the finished catheter 100 comprises a polymer jacketing array 108 and no reinforcement layer 106

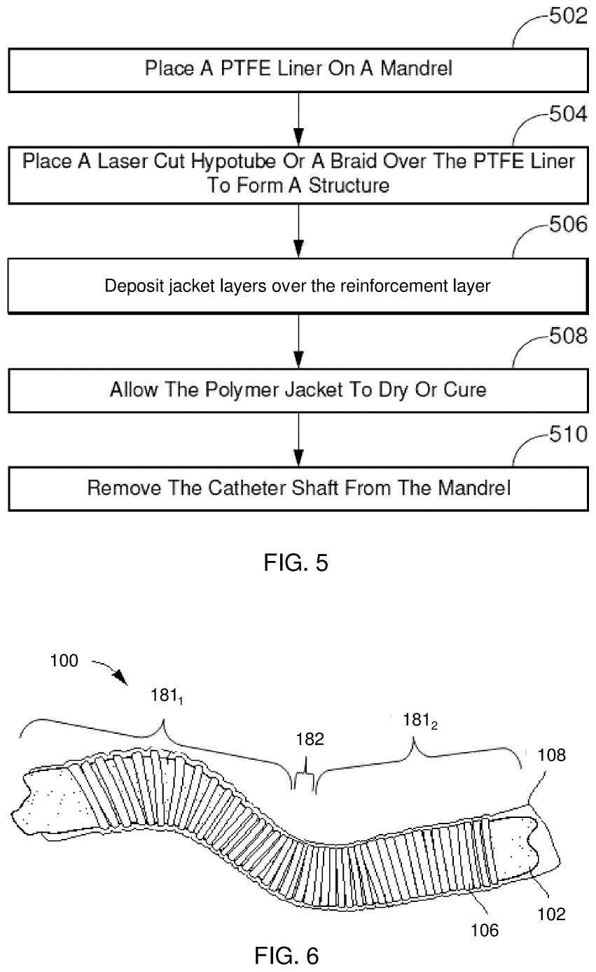

FIG. 5 shows a flowchart illustrating a printing/deposition process for manufacturing the catheter shaft 100 according to embodiments. According to embodiments, the catheter shaft 100 is manufactured using a deposition process of different materials for the shaft, e.g., using a deposition nozzle with an atomizer configured to deposit the materials to form the catheter shaft 100. In some embodiments, the deposition device comprises a focusing mechanism which allows selection of the application width. In some embodiments, the deposition device further comprises a processor. The processor is configured to control the deposition device so that the deposition of material can be coordinated with the usage of the deposition device. In some embodiments, the materials may include, but are not limited to, Polytetrafluoroethylene (PTFE (PTFE-lined catheters)), perfluoroalkoxy alkanes (PFA), fluorinated ethylene propylene (FEP), etc. The catheter shaft 100 then goes through a solution-based process to create a thin, flexible array of polymer jacketing layers that can comprise different materials and be characterized by varying wall thicknesses and hardness value materials along the shaft axis. This process enables the creation of the catheter shaft 100 with high flexibility, pushability, and torquability, and reduced scrap rate. In some embodiments, the solution-based printing process creates the jacketing array 108 on a reinforcement layer mounted on a mandrel, e.g., with an inner liner layer disposed around the mandrel within the tube of the reinforcement layer. In some embodiments, the process fills the slit area/volume with polymer by less than 20% of its volume/area. The liner layer 102 can be bonded to the jacketing array 108 without heat and pressure. The process can also create a covalent bond connection on the reinforcement layer 106 between the metal surface and the polymeric material, e.g., with surface treatment such as a polymeric primer.

In embodiments, the process as illustrated in FIG. 5 comprises at least the following steps. At step 502, a synthetic polymer liner is 102 placed on a mandrel 104. In some embodiments, the mandrel 104 is made of a silver-plated copper core. In some embodiments, the synthetic polymer liner 102 is Polytetrafluoroethylene (PTFE), which is a synthetic fluoropolymer of tetrafluoroethylene. At step 504, the reinforcement layer 106, e.g., in the form of a coil, a braid, or a hypotube, is placed on the PTFE liner provided on the mandrel 104, thereby forming a structure that is used for producing the polymer jacket array 108. At step 506, an array of polymeric jacket layers are deposited over the reinforcement layer 106 and cured at step 508. At step 510, the catheter shaft 100 is removed from the mandrel 104.

FIG. 6 shows a non-limiting example, in cutaway view, of a catheter shaft 100 according to embodiments. The catheter shaft 100 of FIG. 6 comprises a polymeric liner layer 102. In some embodiments, the liner layer 102 may comprise PTFE. The liner layer 102 of FIG. 6 is surrounded by a reinforcement layer 106 comprising a series of coils. In FIG. 6, the catheter shaft 100 comprises two longitudinal sections 181 separated by a transition section 182. The transition section 181 has a length of at least 5 mm and not more than 30 mm. In some embodiments, the two longitudinal sections 181 are characterized by different respective hardness values, and the transition section is characterized by a gradation of hardness values between the different respective hardness values. In some embodiments, the two longitudinal sections 181 comprise different respective jacket-layer material compositions. The transition section 182 comprises gradated intermediate blends of the different jacket-layer material formulations. In some embodiments, the two longitudinal sections 181 comprises different respective jacket-layer material compositions each characterized by a different respective hardness value, while the transition section 182 comprises gradated intermediate blends of the different jacket-layer material formulations and is characterized by a gradation of hardness values between the different respective hardness values. In some embodiments, the maximum hardness value of any of the sections 181 of the catheter shaft 100 is 80 Shore A. In some embodiments, the maximum hardness value of any of the sections 181 of the catheter shaft 100 is 40 Shore A.

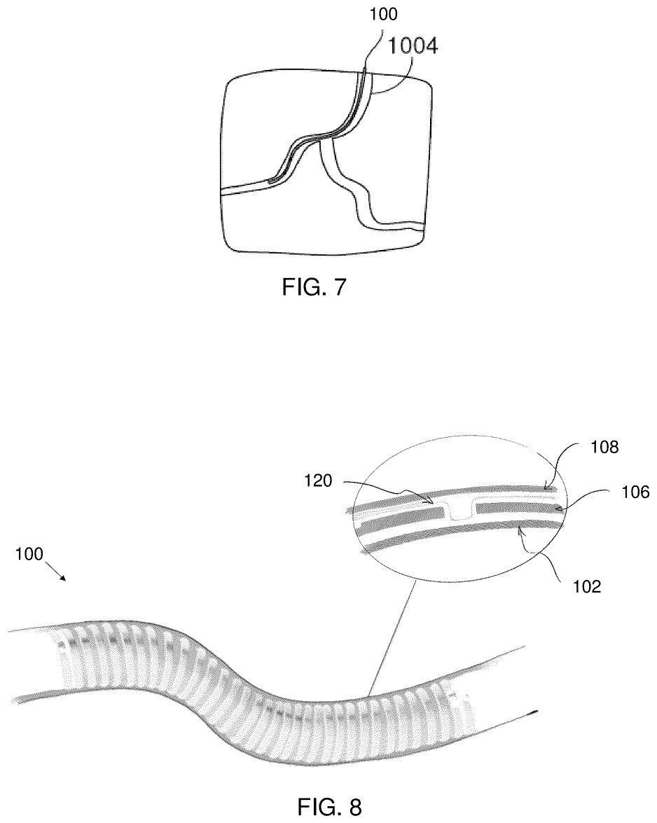

FIG. 7 illustrates a use case for a catheter shaft 100 according to any of the embodiments disclosed herein, showing a catheter shaft 100 positioned inside a first surgical path 1004 at one position.

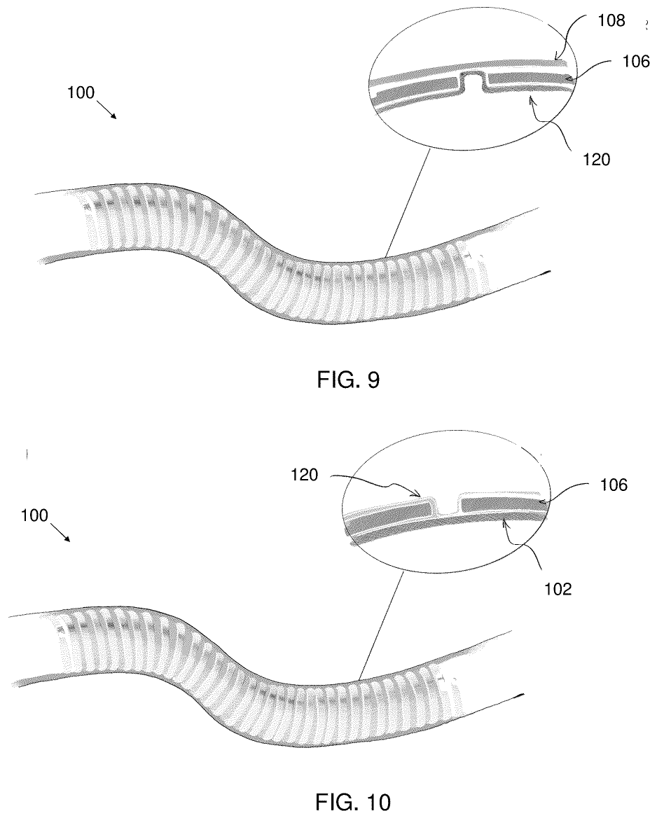

In embodiments, a polymer binding layer can be provided to improve and/or or ensure adherence of a polymer layer, e.g., a liner layer 102 or the outer polymeric jacketing array 108, to a metal-containing reinforcement layer 106. Non-limiting examples of such a binding layer 120 are shown in FIGS. 8, 9 and 10. In the example of FIG. 8, the binding layer 120 is applied between the polymeric jacketing array 108 and an outer surface of the reinforcement layer 106, but the binding layer 120 is shaped and arranged to also contact the inner polymer liner 102, e.g., between rings of the coil. In the example of FIG. 9, the binding layer 120 is applied on the inside of the reinforcement layer 106, and is shaped and arranged to also contact the outer polymeric jacketing array 108, e.g., through or between the rings of the coil of the reinforcement layer 106. In the example of FIG. 10, the binding layer 120 is applied on the outside of the reinforcement layer 106, and is shaped and arranged to also contact the inner polymer liner 102, e.g., between rings of the coil of the reinforcement layer 106.

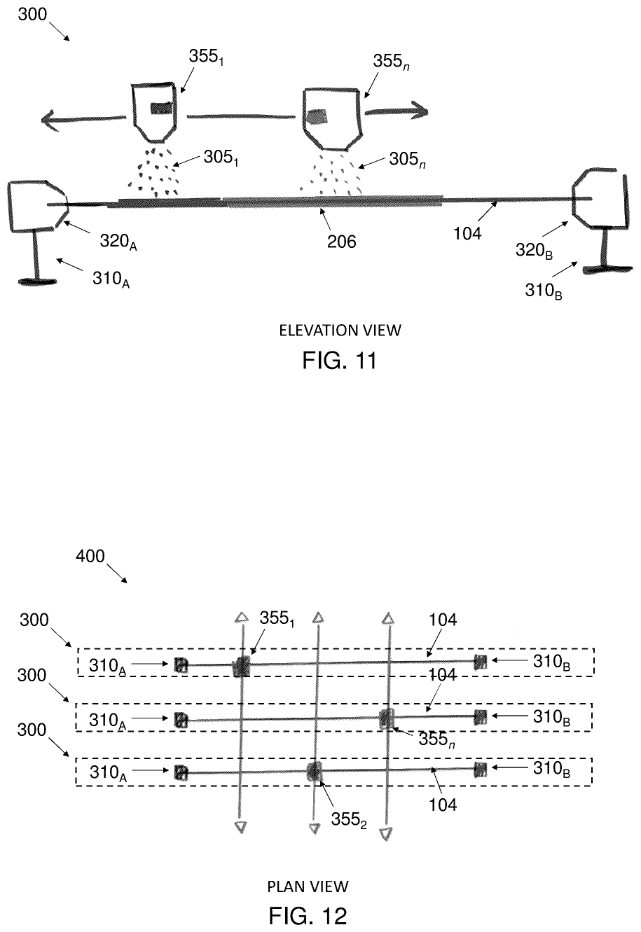

FIGS. 11 and 12 illustrate an exemplary production system for producing catheter shafts 100 according to any of the embodiments disclosed herein. The production system presented here overcomes disadvantages of current catheter shaft production methods in which catheter shafts are produced in multiple sections like telescopes.

FIG. 11 schematically illustrates a coating line 300 in which a mandrel 104 is suspended between two end posts 310A, 310B. The end posts 310A, 310B can include respective cranks or motors 320A, 320B for spooling the ends of the mandrel 104, and/or for ensuring a desired level of tautness in the mandrel 104. According to some embodiments, a liner, e.g., inner polymer liner 102 and/or a binding layer 120, is applied on the mandrel 104. The application of the liner layer 102 can be, for example, by polymer deposition using any one of n nozzles 355₁ . . . 355ₙ. Additionally or alternatively, the mandrel 104 can be mechanically threaded through a provided liner layer 102. Additionally or alternatively, the mandrel (without or without liner) can be mechanically threaded through a reinforcement layer 106. Any one or more of the nozzles 355₁ . . . 355ₙ can be used to deposit one or more layers of a polymeric jacketing array 108. According to some embodiments, the polymeric jacketing array 108 can be produced on the mandrel 104 by the coating line 300 without a reinforcement layer 106, i.e., to produce a polymeric tube comprising an array of polymeric jacket layers.

Different nozzles 355₁ . . . 355ₙ can be used to deposit different respective materials 305₁ . . . 305ₙ. Different materials 305₁ . . . 305n can be deposited in different thicknesses. Additionally or alternatively, any given material can be deposited in different thicknesses at different locations along the length of the mandrel 104, i.e., along the length of the catheter shaft 100. Different materials 305₁ . . . 305ₙ can have different hardness values. In embodiments, differential hardness values along the length of the catheter shaft 100 can be from any combination of different materials and different thicknesses. The different thicknesses can be achieved by depositing coating layers with different thicknesses, and/or by depositing different numbers of coating layers. Differences in thicknesses can be selected to be larger than a typical ±10% tolerance. In an illustrative example, each deposition layer is between 1 and 50 microns thick each, or between 1 and 20 microns, or between 1 and 5 microns. Any portion of the catheter shaft can have at least 5 layers and up to 100 layers. In some examples, a catheter shaft or a portion thereof has between 5 and 50 layers, or between 10 and 30 layers. The total thickness of the deposition layers can be between 25 and 200 microns, or between 50 and 150 microns.

In some embodiments, the materials 3051 . . . 305n can be selected to have different coefficients of friction. In a non-limiting example, the material 305 of an innermost jacket layer has a different coefficient of friction than the material 305 of an outermost jacket layer.

FIG. 12 schematically illustrates shows a production system 400 which includes multiple, parallel (i.e., simultaneous and/or physically parallel) coating lines 300. Any number of nozzles 355 can be deployed, with nozzles being configured for moving between coating lines 300.

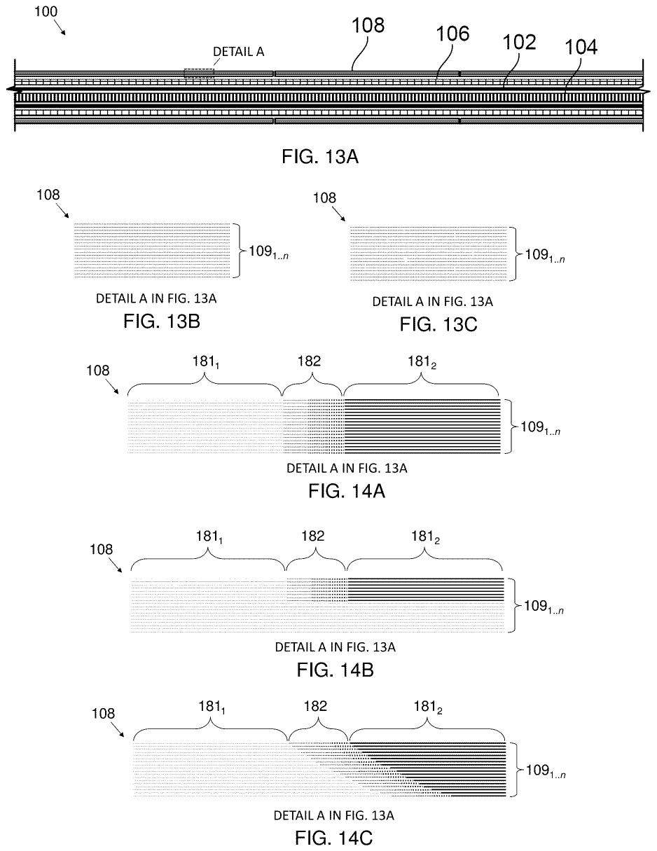

FIG. 13A shows an exemplary catheter shaft 100 according to embodiments, surrounding an elongated mandrel 104, i.e., during a production process. The catheter shaft 100 comprises a liner layer 102 disposed over the mandrel 104, a reinforcement layer 106 disposed over the liner layer 102, and a jacketing array 108 of multiple polymeric jacket layers 109 formed over the reinforcement layer 106. Any of the multiple polymeric jacket layers 109, shown in greater detail in FIGS. 13B and 13C can be formed by an additive manufacturing technique of polymer deposition, involving application of entrained polymeric particles. In some embodiments, all of the multiple polymeric jacket layers 109 are formed by one or more of the polymer deposition technologies. In some embodiments, none of the jacket layers 109 are formed by extrusion or injection molding. The drops can be in a melted or dissolved state, or otherwise entrained in a fluid, and can comprise one or suitable polymeric materials, including, but not exhaustively: polytetrafluoroethylene (PTFE), polyether-block-amide (PEBAX) and a polyurethane. When thermal spraying is used, the selected polymer can be one with a melt flow index range of 2 to 30 g (i.e., tested for 10 min at 190-210° C. in a 3-10 kg sample). Drops are delivered having a diameter in the range of 1 to 200 microns, or 1 to 100 microns, or 1 to 50 microns, or 1 to 10 microns. In some embodiments, the liner layer 102 is formed by an additive manufacturing technique involving deposition polymeric particles, i.e., the same as, or similar to, the technique(s) as used for the polymeric jacket layers 109.

In some embodiments, as shown schematically in FIG. 13C, some or all of the polymeric jacket layers 109 are formed as multiple longitudinal segments. The multiple longitudinal segments can be contiguous or, as illustrated, not contiguous.

Two or more adjoining longitudinal sections can be characterized, for example, by different hardness values or by different composition (including, without limitation, different polymers, different concentrations, different colors or different radiodensities), i.e., where the adjoining longitudinal sections comprise different respective jacket-layer material formulations. Adding a colorant or radiopaque material to a segment can be accomplished by any one of several methods, including, and not exhaustively: dissolving the colorant or radiopaque material in the polymeric solution; synthesizing the colorant or radiopaque material into the polymeric molecule to form a synthesized polymer molecule such as, for example, a dye-grafted polyurethane; and creating an emulsion including the colorant or radiopaque material to pass through the deposition nozzles or jets. The latter option may include adding a stirring or vibrating option before the nozzles or jets. In some applications, gold and/or barium is added to a jacket-layer composition, e.g., for visibility under fluoroscopy.

In some embodiments, a catheter shaft 100 comprises two adjoining longitudinal sections comprising different respective jacket-layer material formulations. In a first example, a longitudinal segment comprises a harder material that provides greater radial flexibility together with greater longitudinal stiffness. In a second example, a longitudinal segment comprises a softer, i.e., less hard, material that provides greater radial stiffness together with greater longitudinal flexibility. The adjoining longitudinal sections can be connected by a material transition zone between the adjoining longitudinal sections, where the transition zone is characterized, for example, by one or more intermediate blends of the different hardnesses and/or different jacket-layer material formulations. In embodiments, the length of such a transition zone can range from 5 mm to 300 mm, e.g., between 5 and 20 mm, between 5 and 30 mm, or between 100 and 300 mm, or any other intermediate range.

Referring to FIGS. 14A, 14B and 14C, illustrative examples of transition sections 182 in jacket layers 109 are shown. In these three figures, the relative thickness of the segments 181 is intended to convey a material property such as hardness, composition, and/or thickness, and not necessarily thickness. In the example of FIG. 14A, the transition section 182, where the material property distinguishing the two longitudinal segments 181 transitions between the two longitudinal segments 181 in any combination of steps and continuous change, is the same for each of the jacket layers 109. In the example of FIG. 14B, the material property or properties do not change along the length of the innermost jacket layers 109, while the outermost layers comprise the two longitudinal segments 181 separated by the transition section 182. In the example of FIG. 14C, the transition sections 182 of the respective jacket layers 109 are staggered longitudinally. Other examples of transition section 182 placement, layer differentiation and staggering, not shown, can be used, including combinations of any the features of FIGS. 14A, 14B and 14C.

Various factors enter into the design choices such as the number, thickness and composition of the polymeric jacket layers 109 (or of segments thereof). Non-limiting examples of such factors include the desired characteristics of the catheter shaft 100 such as dimensions (e.g., thicknesses), surface hardnesses, surface roughnesses, flexibility and/or ductability. A catheter shaft 100 according to embodiments can be designed to have a tensile strength in the range 20-60 MPa.

In embodiments, the jacketing layer 108 includes at least 5 polymeric jacket layers 109. In some embodiments, the jacketing layer 108 includes at least 10, or at least 15, or at least 20, or at least 25, or at least 30, or at least 35, or at least 40, or at least 45, or at least 50, or at least 75, or at least 100, or at least 125, or at least 150 polymeric jacket layers 109.

In embodiments, each of the polymeric jacket layers 109 has a minimum thickness of at least 0.6 microns and a maximum thickness of at most 10 microns. In embodiments, each of the polymeric jacket layers 109 has a minimum thickness of at least 0.8 microns and a maximum thickness of at most 6 microns. In some embodiments, each of the polymeric jacket layers 109 has a minimum thickness between 0.5 microns and 2 microns, or between 0.6 microns and 1.5 microns, or between 0.8 microns and 1.2 microns, all ranges cited herein being inclusive; in such embodiments, each of the polymeric jacket layers 109 has a maximum thickness between 5 microns and 10 microns, or between 5 microns and 8 microns, or between 6 microns and 8 microns, such that a suitable range of thicknesses can include any combination of the foregoing minima and maxima.

In embodiments, the jacketing array 108 has a total thickness between 5 microns and 200 microns, or between 15 microns and 100 microns, or any other intermediate range. The thickness of the jacketing array 108 can be between 0.01% and 10% of the outer diameter of the catheter shaft 100, or between . 1% and 1% of the outer diameter of the catheter shaft 100, or any other intermediate range. In an example, a jacketing array 108 has a thickness of not more than 0.2% of the outer diameter of the catheter shaft 100, and optionally not less than 0.02% or 0.01%. In embodiments, the inner diameter of the catheter shaft 100 can be between 100microns and 5 millimeters, or any range therebetween, or higher or lower, e.g., less than 100 microns.

The catheter shaft 100, and/or any one or more of its polymeric jacket layers 109, can have a hardness of less than 80 Shore A, or less than 60 Shore A, or less than 40 shore A. In some embodiments, the liner layer 102 has a higher hardness value than at least one of the jacket layers 109 or a higher hardness value than any, i.e., all, of the jacket layers 109. In some embodiments, two or more adjoining segments of the jacketing array 108, or of one or more its polymeric jacket layers 109, can have different hardness values.

According to embodiments, a system for manufacturing catheter shafts according to embodiments can include one or more holder assemblies for mounting thereupon a mandrel, and one or more nozzles or jets for deposition of one or more polymeric materials. In some embodiments, the system includes one or more air tubes or the equivalent for drying drops deposited onto the mandrel. In some embodiments, the system includes cutting equipment for cutting and trimming catheter shafts. The system can also include various control circuitry, including an imaging system.

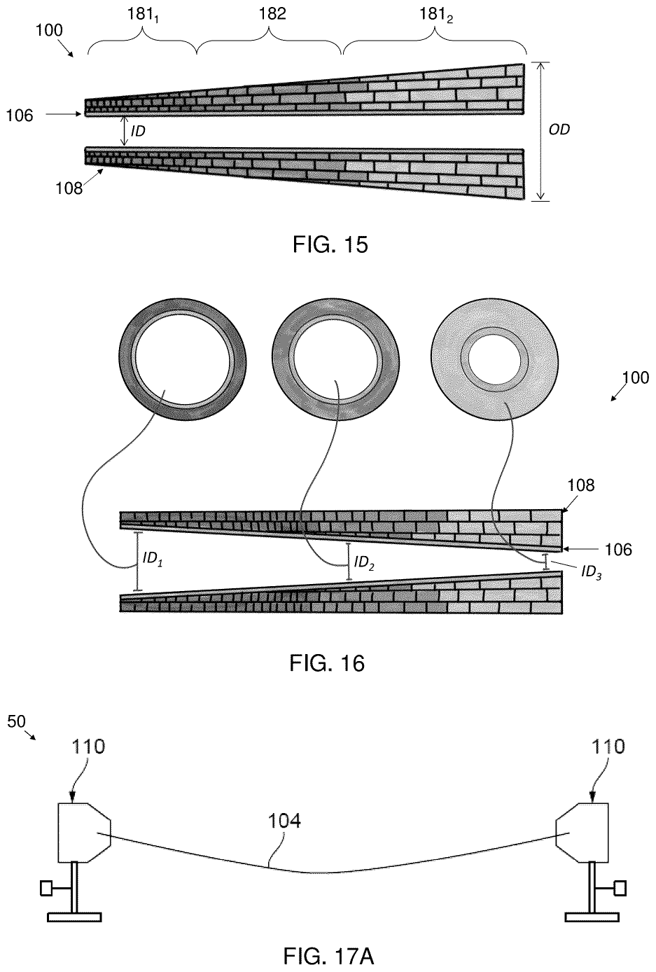

In some embodiments, as schematically illustrated in FIGS. 15 and 16, the inner diameter and/or outer diameter of a catheter shaft 100 according to embodiments can vary along its length. FIG. 15 schematically illustrates a catheter shaft 100 having a constant inner diameter ID and a variable outer diameter OD. FIG. 16 schematically illustrates a catheter shaft 100 having a variable inner diameter ID and a constant outer diameter OD. In another example, not shown, both the inner and outer diameters can be variable. In other examples, also not shown, the variability of the inner and/or outer diameter need not be linear or monotonic.

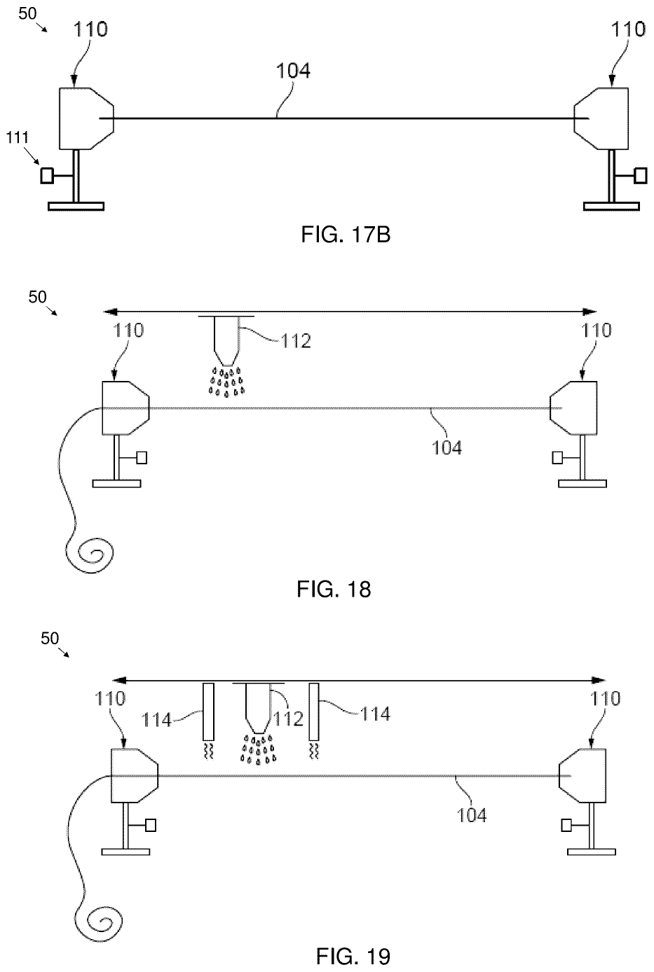

FIGS. 17A and 17B are schematic diagrams of selected components of a system 50 for manufacturing a catheter shaft 100 according to embodiments. An elongated mandrel 104 is arranged to extend between two holder assemblies 110. FIG. 17A shows slack in the mandrel 104, while in FIG. 17B, the slack has been taken out by a tensioning mechanism in one or more of the holder assemblies 110; the tensioning can be manual (i.e., a crank 111), or at least one of the holder assemblies 110 can include a motor.

In embodiments, the outer diameter of a mandrel 104 is selected in accordance with a desired internal diameter of the catheter shaft 100 to be produced thereupon.

For example, if the desired internal diameter of a catheter shaft 100 is less than 100 microns, then the outer diameter of a mandrel 104 is selected to be less than 100 microns. The mandrel 104 can be of any practical length; in non-limiting examples, a mandrel is between 50 and 200 cm in length. Examples of suitable materials for the mandrel 104 include, and not exhaustively, silver-coated copper, silver-coated aluminum, and PTFE reinforced with glass fibers. It can be desirable to provide a mandrel 104 that can be non-destructively removed from inside a catheter shaft, and to this end the outer surface of the mandrel 104 can be characterized by an average surface roughness of as little as 15 microns and/or not more than 45 microns. Further, a mandrel 104 can be characterized by a ductility of at least 25%.

As shown schematically in FIG. 18, a system 50 can comprise one or more nozzles 112. The term ‘nozzle’ is used herein to mean any element that can spray, disperse, drip or deposit drops of a solution or suspension comprising polymeric materials. The nozzle 112 is configured to move between the holder assemblies 110. The nozzle 112 is disposed above the holder assemblies 110 and adapted to move parallel to the mandrel 104 that extends between the holder assemblies 110. In some embodiments, not illustrated, the system 50 includes arrangements to rotate the nozzle(s) 112 around the mandrel 104 and its coverings and coatings. In some embodiments, the system 50 includes arrangements to rotate the mandrel 104.

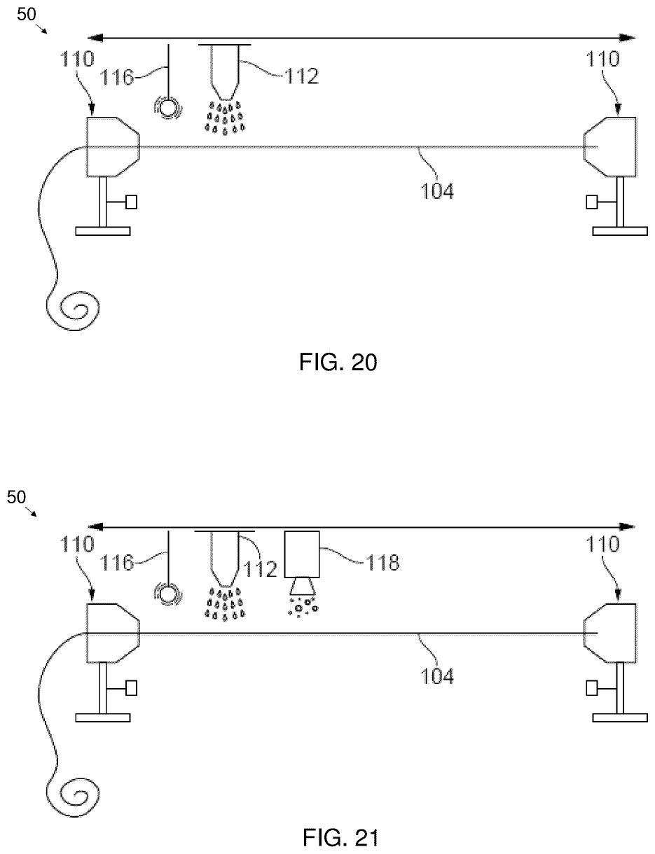

As shown schematically in FIG. 19, a system 50 can comprise one or more air tubes 114 or equivalent arrangements for drying, e.g., hot-air drying, for evaporating a solvent contained in the drops. In an example, the air tubes 114 are configured to blow air at a temperature between 20° C. and 100° C. and/or with humidity under to 20%. Additionally or alternatively, a system 50 can include one or more radio frequency (RF) dryers 116, as shown in FIG. 20. Like the air-drying tube(s) 114, the RF dryer(s) 116 can be disposed in line with and adjacent to the one or more nozzles 112, and is configured to dry the polymer deposited over the mandrel 104 (or over previously applied layers).

In some applications, as shown schematically in FIG. 21, the system 50 includes and makes use of a plasma jet 118 to deposit polymer, entrained in the plasma fluid, over the mandrel 104 or over a previously applied layer 109. The polymer can be introduced into the plasma jet 118 as dry particles or as a liquid, i.e., a suspension or solution. In some applications, the system 50 includes and make use of a plasma jet 118 to perform surface activation of the outer surface of the reinforcement layer 106 before depositing the first jacket layer 109. In some embodiments, the plasma jet 118 uses surface corona discharge-induced plasma deposition. In some embodiments, the reinforcement layer 106 undergoes another form of surface activation or treatment, e.g., chemical before polymer deposition.

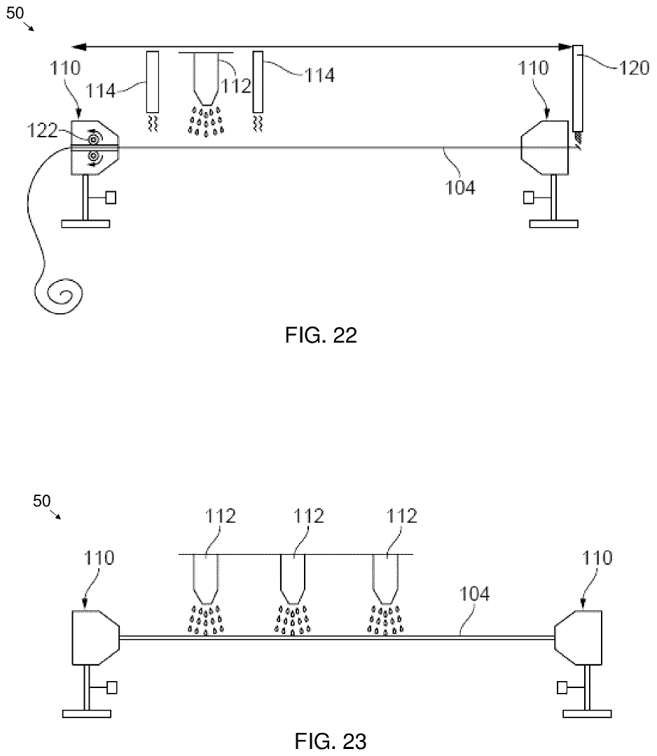

In embodiments, it can be desirable to produce catheter shafts 100 in a continuous process rather than in small batches. In a non-limiting example illustrated schematically in FIG. 22, at least one of the holder assemblies 110 includes a motor 122 for tensioning the mandrel 104 and/or for advancing the mandrel 104 for continuous or semi-continuous production. In the example of FIG. 22, the system 50 includes a cutting device 120 such as a guillotine for cutting the catheter shaft 100, e.g., with some or all layers applied, to a desired length.

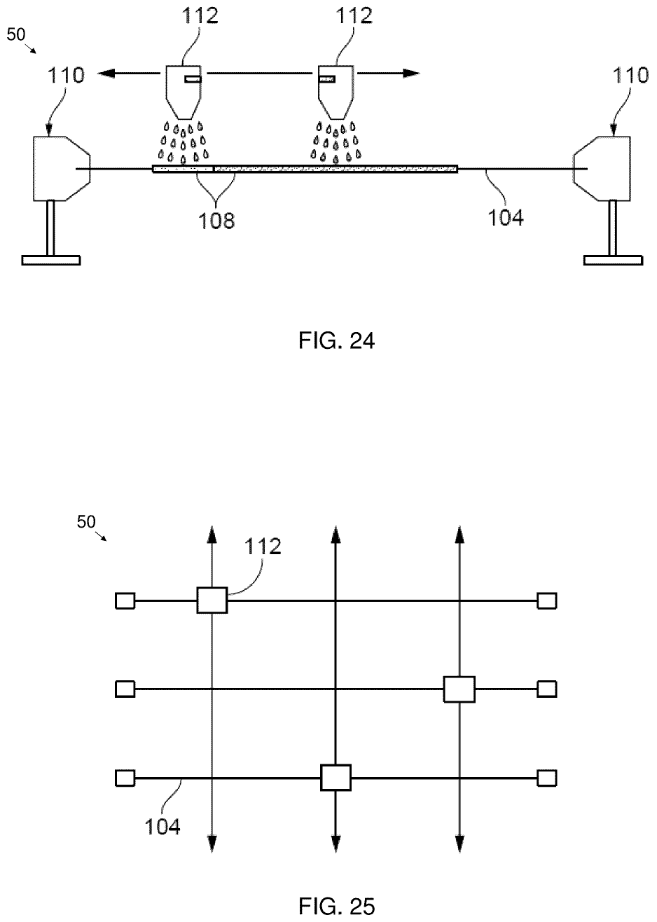

FIGS. 23, 24 and 25 illustrate systems 50 which are not limited to use of a single mandrel 104 coated by a single nozzle 112 (or single plasma jet 118, not shown), and instead employ multiple mandrels 104, e.g., for producing multiple catheter shafts 100 in parallel. FIG. 24 schematically shows an example of using multiple nozzles 112 to produce a catheter shaft 100 comprising different polymeric materials or material compositions characterized by different hardnesses in adjoining segments. The different nozzles 112 can also be used for changing composition, hardness and/or thicknesses between the jacket layers 109 that make up the jacketing layer 108. FIG. 25, like FIG. 12, schematically illustrates a top view showing an arrangement of a plurality of holder assemblies 110 supporting mandrels 104 and nozzles 112 moving in parallel and above the mandrels 104. This is an illustrative example of how systems 50 can be designed to produce any reasonable number of catheter shafts in parallel, using desired numbers of nozzles 112, along with a desired number of air-drying tubes 114, RF dryers 116, plasma jets 118, and so on.

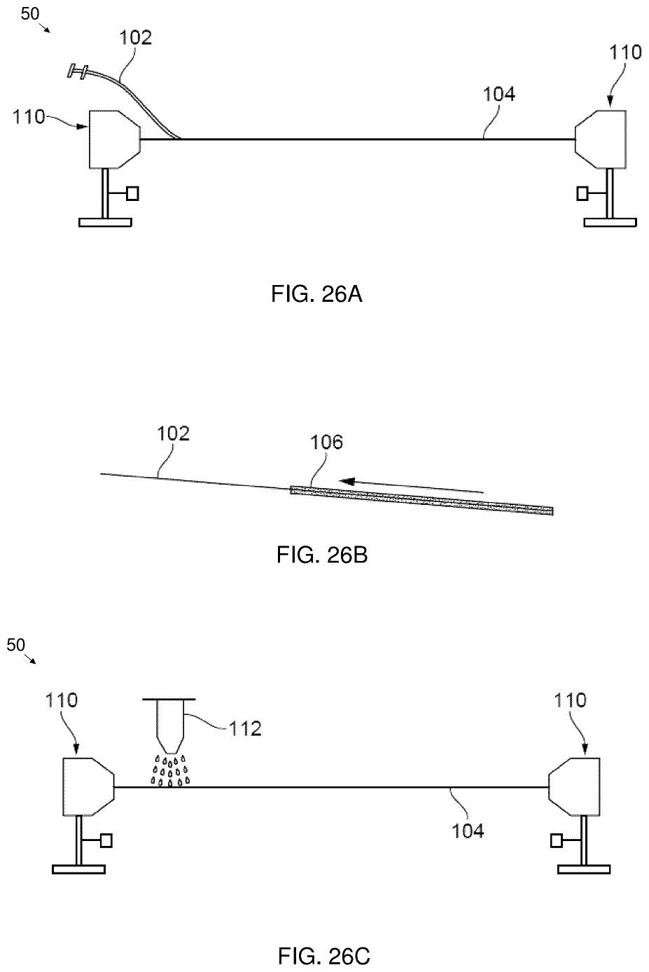

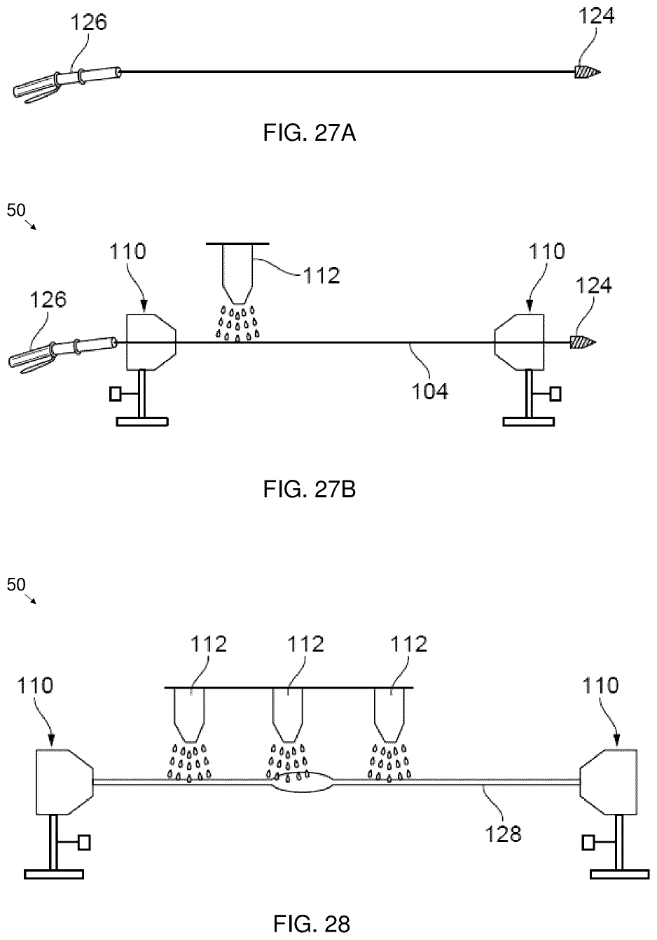

FIGS. 26A, 26B and 26C schematically illustrate selected steps of a method for producing catheter shafts 100 according to any of the embodiments disclosed herein. FIG. 26A illustrates providing a pre-fabricated, e.g. extruded, liner layer 102 over a mandrel 104. In other examples, the liner layer 102 is formed by deposition by using the system components in the same way as (or similar to) for depositing polymeric jacket layers 109. In yet other examples, the liner layer 102 is directly extruded over the mandrel 104. As illustrated in FIG. 26B, a reinforcement layer 106 is disposed over liner layer 102. Reinforcement layers 106 are commonly formed from metal or metal alloys, and in some case from polymers, in the form of braids, laser-cut hypo-tubes, or coils. The reinforcement layer 106 can be drawn over the mandrel 104 and liner layer 102, or can be deposited directly onto the liner layer. In some embodiments, the reinforcement layer 106 can be subjected to a surface treatment, e.g., a chemical surface treatment, before application of the jacketing layers 109, for better adhesion and bonding, or can include a bonding layer such as that shown in FIGS. 8, 9 and 10. FIGS. 27A and 27B schematically illustrate the attachment of a tip 124 and a handle 126 to the mandrel 104 prior to its installation on the holder assemblies 110.

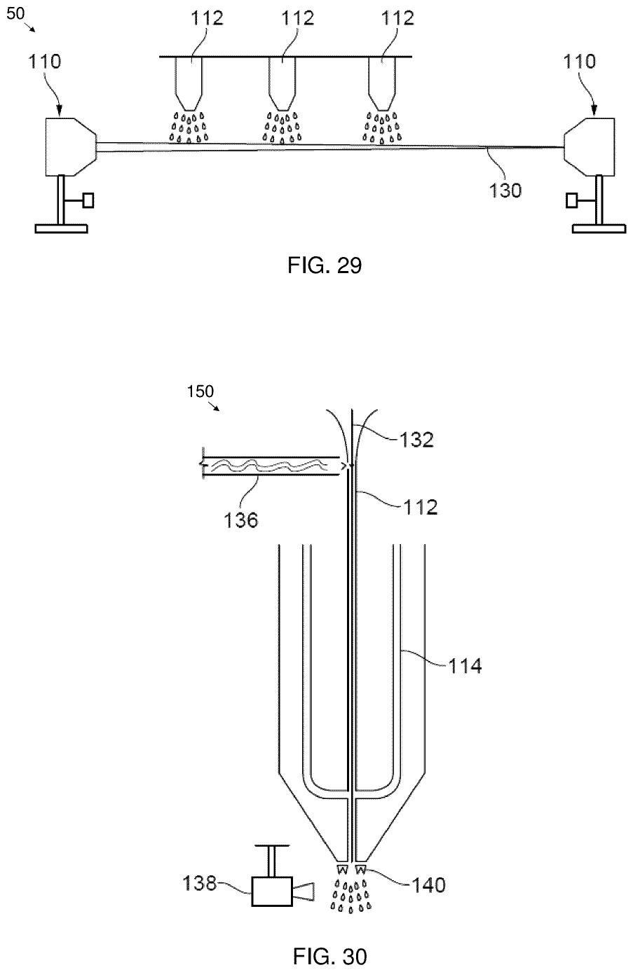

In embodiments, it can be desirable to employ a mandrel characterized by an irregular shape, i.e., a shape that can be used to form a catheter shaft 100 that is not a simple elongated cylinder. Examples shown in FIGS. 28 and 29 include a balloon type mandrel 128 and a cone type mandrel 130, respectively. The cone-type mandrel 130 of FIG. 29 can be used, for example in producing the variable interior-diameter catheter shaft 100 of FIG. 16.

Referring to FIG. 30, a system 50 can comprise an integrated tube 150 comprising a nozzle 112 having a magnetic needle 132 extending therethrough, one or more air tubes 114 fluidly coupled to the nozzle 112 and one or more solvent tubes 136 fluidly coupled to the nozzle 112 via a one-way valve. The magnetic needle 132 is configured for cleaning the nozzle 112. The system 50 of FIG. 30 further comprises an imaging system 138 to enable real time control of the deposition. The integrated tube can further include an ultrasonic module 140.

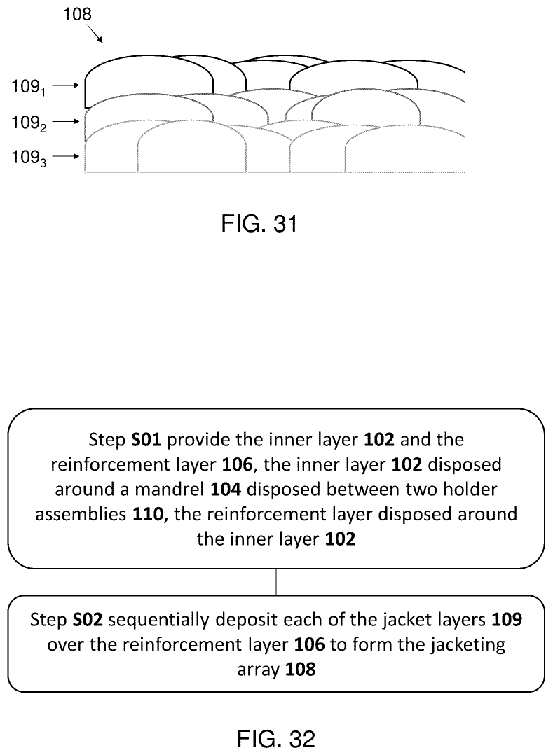

In embodiments, drops deposited onto a mandrel 104 or on a preceding layer 109 tend to flatten upon impact and/or upon the surface of the substrate before the next layer is applied. In some implementations, the drops coalesce and form a flat or nearly flat surface; this can depend upon factors such as, and not exhaustively:
viscosity, concentration of polymer in the deposited drops, curing time (e.g., drying time) and time between application of subsequent layers. In an example, a solution comprising between 3% and 10% polymer in a solvent having a viscosity in the range of 1 to 10 Cp, or 5-10 Cp, a ‘bumpy’ surface can be evident on on the outer surface of a catheter shaft 100. In an example shown in FIG. 31, each of the three uppermost jacket layers 109₁, 109₂, 109₃ of a jacketing array 108 retain a bumpy upper surface as the polymer dries and solidifies before completely coalescing to form a flat surface. In embodiments, the catheter shaft 100 has an average surface roughness of at least 0.1 microns and not more than 5 microns.

Referring now to FIG. 32, a method is disclosed for manufacturing, by polymer deposition, the catheter shaft 100 according to any of the embodiments disclosed herein. As illustrated by the flow chart in FIG. 32, the method comprises at least the two method steps S01 and S02:

Step S01 includes providing the inner layer 102 and the reinforcement layer 106, the inner layer 102 disposed around a portion of an elongated mandrel 104 disposed between two holder assemblies 110, and the reinforcement layer 106 disposed around at least a portion of the inner layer 102. In some embodiments, providing the inner layer 102 includes forming the inner layer 102 by depositing a polymeric coating over the mandrel 104.

Step S02 includes sequentially depositing each of the jacket layers 109 over the reinforcement layer 106 to form the jacketing array 108.

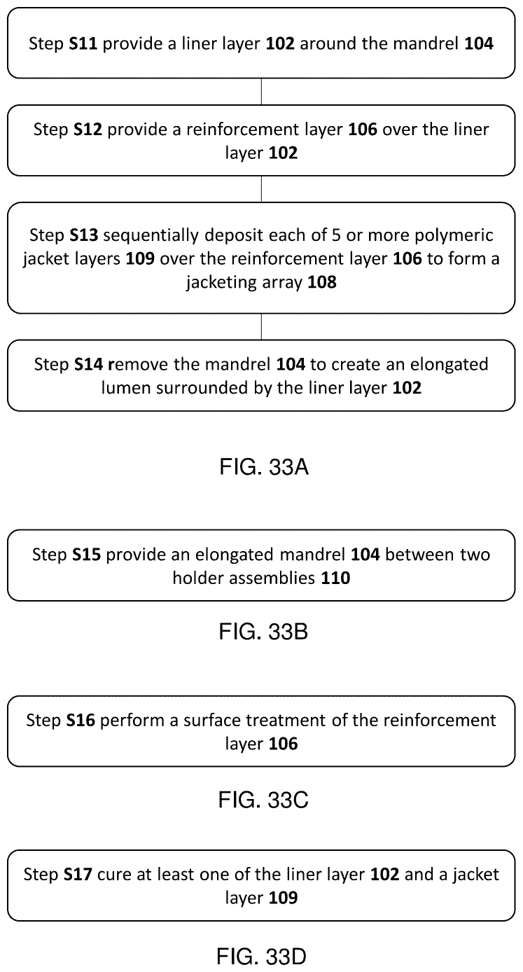

Referring now to FIG. 33A, a method is disclosed for manufacturing a catheter shaft 100. As illustrated by the flow chart in FIG. 33A, the method comprises at least the 5 method steps S11, S12, S13, S14 and S15:

Step S11 includes providing a liner layer 102, disposed around a mandrel 104. In some embodiments, providing the liner layer 102 includes forming the liner layer 102 by depositing the liner layer 102 over the mandrel 104. In some embodiments, an outer surface of the mandrel 104 is characterized by an average surface roughness of 15 to 45 microns. In some embodiments, the mandrel 104 comprises one of: silver-coated copper, silver-coated aluminum, and PTFE reinforced with glass fibers. In some embodiments, the mandrel 104 has a ductility of at least 25%.

Step S12 includes providing a reinforcement layer 106 over the liner layer 102.

Step S13 includes sequentially depositing each of 5 or more polymeric jacket layers 109 over the reinforcement layer 106 to form a jacketing array 108, each jacket layer 109 having a minimum thickness of at least 1 micron and a maximum thickness of at most 5 microns.

In some embodiments, the sequentially depositing comprises, for at least one of the jacket layers 198, forming first and second longitudinal sections 181 characterized by different respective hardness values and a transition section 182 therebetween having a length of at least 5 mm and not more than 30 mm, the transition section 182 being characterized by a gradation of hardness values between the different respective hardness values. In some embodiments, the sequentially depositing comprises, for at least one of the jacket layers 109, forming first and second longitudinal sections 181 comprising different respective jacket-layer material compositions and separated by a transition section 182 having a length of at least 5 mm and not more than 30 mm, the transition section 182 comprising gradated intermediate blends of the different jacket-layer material formulations. In some embodiments, the sequentially depositing comprises, for at least one of the jacket layers 109, forming first and second longitudinal sections 181 separated by a transition section 182 having a length of at least 5 mm and not more than 30 mm, the first and second longitudinal sections 181 comprising different respective jacket-layer material compositions each characterized by a different respective hardness value, wherein the transition section 182 comprises gradated intermediate blends of the different jacket-layer material formulations and is characterized by a gradation of hardness values between the different respective hardness values.

In some embodiments, the sequentially depositing comprises sequentially depositing each of 10 or more polymeric jacket layers 109, each having a minimum thickness of at least 1 micron and a maximum thickness of at most 5 microns. In some embodiments, the sequentially depositing comprises sequentially depositing each of 25 or more polymeric jacket layers 109, each having a minimum thickness of at least 1 micron and a maximum thickness of at most 5 microns.

Step S14 includes removing the mandrel 104 to create an elongated lumen surrounded by the liner layer 102.

In some embodiments, as illustrated by the flowchart in FIG. 33B, the method can additionally comprise the method step S15, which is performed before Step S11.

Step S15 includes providing the elongated mandrel 104 between the two holder assemblies 110.

In some embodiments, as illustrated by the flow chart in FIG. 33C, the method can additionally comprise the method step S16:

Step S16 includes performing a surface treatment of the reinforcement layer 106. If included in carrying out the method, Step S16 is carried out before Step S13.

In some embodiments, as illustrated by the flow chart in FIG. 33D, the method can additionally comprise the method step S17:

Step S17 includes curing at least one of the liner layer 102 and a jacket layer 109, wherein the curing includes at least one of drying, heating, exposing to a selected radio frequency (RF), and irradiating with UV or IR light.

In some embodiments, the manufactured catheter shaft 100 has an average surface roughness of at least 0.1 microns and not more than 5 microns.

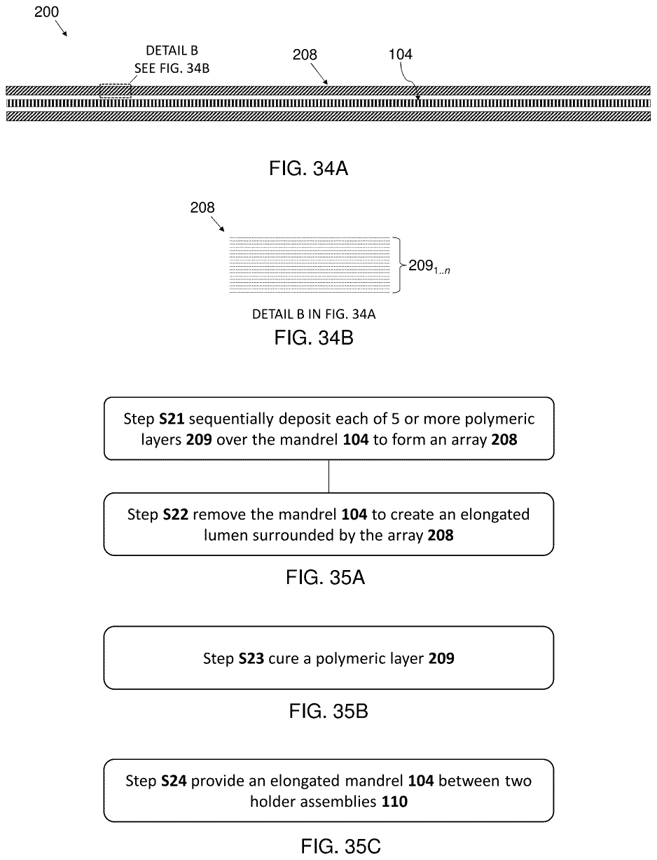

We now refer to FIGS. 34A and 34B. A polymeric tube 200 according to embodiments comprises an array 208 of polymeric layers 209 formed by deposition onto a mandrel 104. The polymeric tube 200 is structurally and compositionally equivalent to a catheter shaft 100 according to the embodiments disclosed hereinabove, mutatis mutandis, i.e., without a reinforcement layer and in some designs without an inner layer. Thus, any of the systems 50 disclosed, e.g., in FIGS. 17A, 17B, 18, 19, 20, 21, 22, 23, 24, 25, 26A, 26C, 27B, 28, and 29, as well as the integrated tube apparatus 150 of FIG. 30, can be used to produce a polymeric tube 200.

Referring now to FIG. 35A, a method is disclosed for manufacturing a polymer tube 200. In some embodiments, the method is effect to produce a polymeric tube 200 comprising an array 208 of at least 10 polymeric layers 209 each having a minimum thickness of at least 0.5 microns and a maximum thickness of at most 10 microns. As illustrated by the flow chart in FIG. 35A, the method comprises at least the 3 method steps S21, S22 and S23:

Step S21 includes sequentially depositing a plurality of (e.g., 5 or more) polymeric layers 209 over the mandrel 104 to form an array 208 of polymeric layers 209, each of the polymeric layers 209 having a minimum thickness of at least 1 micron and a maximum thickness of at most 5 microns. In some embodiments, the sequentially deposition comprises, for at least one of the polymeric layers 209, forming two adjoining longitudinal sections characterized by different respective hardness values. In some embodiments, the sequentially applying comprises, for at least one of the polymeric layers 209, forming two adjoining longitudinal sections comprising different respective polymeric-layer material formulations. In some embodiments, the sequentially applying comprises sequentially applying each of 10 or more polymeric layers, each having a minimum thickness of at least 1 micron and a maximum thickness of at most 5 microns. In some embodiments, the sequentially applying comprises sequentially applying each of 25 or more polymeric layers, each having a minimum thickness of at least 1 micron and a maximum thickness of at most 5 microns. In some embodiments, an outer surface of the mandrel 104 is characterized by an average surface roughness of 15 to 45 microns. In some embodiments, the mandrel 104 comprises one of: silver-coated copper, silver-coated aluminum, and PTFE reinforced with glass fibers. In some embodiments, the mandrel 104 has a ductility of at least 25%.

Step S22 includes removing the mandrel 104 to create an elongated lumen surrounded by the array 208.

In some embodiments, as illustrated by the flow chart in FIG. 35B, the method can additionally comprise the method step S23.

Step S23 includes curing a polymeric layer 209, wherein the curing includes at least one of drying, heating, exposing to a selected radio frequency (RF), and irradiating with UV or IR light.

In some embodiments, as illustrated by the flow chart in FIG. 35C, the method can additionally comprise the method step S24.

Step S24 includes providing the elongated mandrel 104 between two holder assemblies 110.

In some embodiments, the manufactured polymeric tube 209 has an average surface roughness of at least 0.1 microns and not more than 5 microns.

In embodiments, the disclosed methods for manufacturing polymer tubes 200 and catheter shafts 100 yield improvements in variability of the results. For example, current manufacturing processes including, without limitation, extrusion of relatively soft thermoplastic materials are characterized by variations, i.e., high tolerances, in wall thickness and concentricity. Such variances, often reaching +20% or more, are present between lots and within lots, especially in tubes or catheter shafts of small diameters, e.g., 2 mm or less, or large diameters, e.g., 10 mm or less. The inventors have found that the production methods disclosed herein reliably yield wall thickness and concentricity tolerances of no more than +5%, or no more than ±7%, or no more than ±10%.

### Additional Embodiments

In embodiments, a catheter shaft comprises: (a) a liner layer surrounding an elongated lumen; (b) a reinforcement layer at least partly surrounding the liner layer and arranged coaxially therewith; and (c) a jacketing array at least partly surrounding the reinforcement layer and arranged coaxially therewith, the jacketing array comprising at least 10 polymeric jacket layers each having a minimum thickness of at least 0.5 microns and a maximum thickness of at most 10 microns.

In some embodiments, it can be that at least one of the jacket layers is formed by deposition of drops. In some embodiments, all of the jacket layers can be formed by deposition of drops. In some embodiments, it can be that none of the jacket layers are formed by extrusion or injection molding.

In some embodiments, the catheter shaft can comprise two adjoining longitudinal sections characterized by different hardness values. In some embodiments, the catheter shaft can comprise two adjoining longitudinal sections comprising different respective jacket-layer material formulations. In some embodiments, such the catheter shaft can comprise a material transition zone between the adjoining longitudinal sections, the transition zone being characterized by one or more intermediate blends of the different jacket-layer material formulations.

In some embodiments, each of the jacket layers can have a minimum thickness of at least 0.8 microns and a maximum thickness of at most 6 microns. In some embodiments, the jacketing array can comprise at least 25 jacket layers. In some embodiments, the jacketing array can comprise at least 50 jacket layers.

In some embodiments, the liner layer can have a higher hardness value than at least one of the jacket layers. In some embodiments, the liner layer can have a higher hardness value than any of the jacket layers.

In some embodiments, a ratio of a maximum jacket-layer thickness to a diameter of the catheter shaft can be less than or equal to 0.2%. In some embodiments, the catheter shaft can have an internal diameter of not more than 100 microns. In some embodiments, the catheter shaft can be characterized by an average surface roughness of at least 0.1 microns and no more than 5 microns.

In some embodiments, a method for manufacturing the catheter shaft of any one of the foregoing embodiments can comprise: (a) providing the inner layer and the reinforcement layer, the inner layer disposed around a portion of an elongated mandrel disposed between two holder assemblies and the reinforcement layer disposed around at least a portion of the inner layer; and (b) sequentially depositing each of the jacket layers over the reinforcement layer to form the jacketing array. In some embodiments, providing the inner layer can include forming the inner layer by depositing a polymeric coating over the mandrel.

In some embodiments, a system for manufacturing a catheter shaft of any of the foregoing embodiments can comprise: (a) a plurality of spaced-apart holder assemblies configured to support an elongated mandrel therebetween, at least one of the holder assemblies further configured to apply a tension to the mandrel; and (b) one or more nozzles, arranged to move along a length of the mandrel and deposit one or more of a liner layer and a jacket layer of a jacketing array.

A method is disclosed, according to embodiments, for manufacturing a catheter shaft. The method comprises: (a) providing an elongated mandrel between two holder assemblies; (b) providing a liner layer, disposed around the mandrel; (c) providing a reinforcement layer over the liner layer; (d) sequentially depositing a plurality, or each of 5 or more, or each of 10 or more, polymeric jacket layers over the reinforcement layer to form a jacketing array; and (e) removing the mandrel to create an elongated lumen surrounded by the liner layer.

In some embodiments of the method, providing the liner layer can include one of: (i) forming the liner layer by depositing a polymeric coating over the mandrel, and (ii) providing an extruded or otherwise prefabricated liner layer.

In some embodiments, the method can additionally comprise performing a surface treatment of the reinforcement layer. In some embodiments, the method can additionally comprise curing at least one of the liner layer and a jacket layer. The curing can include at least one of drying, heating, exposing to a selected radio frequency (RF), and irradiating with UV or IR light.

In some embodiments, it can be that each of the sequentially applied jacket layers has a minimum thickness of at least 0.5 microns and a maximum thickness of at most 10 microns when cured.

In some embodiments, the sequentially applying can comprise, for at least one of the jacket layers, forming two adjoining longitudinal sections characterized by different respective hardness values. In some embodiments, the sequentially applying can comprise, for at least one of the jacket layers, forming two adjoining longitudinal sections comprising different respective jacket-layer material formulations.

In some embodiments, the sequentially applying can comprise sequentially applying each of 25 or more polymeric jacket layers. In some embodiments, the sequentially applying can comprise sequentially applying each of 50 or more polymeric jacket layers.

In some embodiments of the method, an outer surface of the mandrel can be characterized by an average surface roughness of 15 to 45 microns. In some embodiments, the mandrel can comprises one (or more) of: silver-coated copper, silver-coated aluminum, and PTFE reinforced with glass fibers. In some embodiments, the mandrel can has a ductility of at least 25%.

In some embodiments, it can be that each of the polymeric jacket layers has a minimum thickness of at least 0.5 microns and a maximum thickness of at most 10 microns. In some embodiments, it can be that the manufactured catheter shaft has an average surface roughness of at least 0.1 microns and not more than 5 microns.

In some embodiments, a catheter shaft produced by the method of any one of the foregoing embodiments can comprise a liner layer, a reinforcement layer, and a jacketing array comprising at least 10 polymeric jacket layers each having a minimum thickness of at least 0.5 microns and a maximum thickness of at most 10 microns.

According to embodiments disclosed herein, a system for producing a catheter shaft comprises: (a) a plurality of spaced-apart holder assemblies configured to support an elongated mandrel therebetween, at least one of the holder assemblies further configured to apply a tension to the mandrel; and (b) one or more nozzles, arranged to move along a length of the mandrel and deposit one or more of a liner layer and a jacket layer of a jacketing array. In some embodiments, it can be that the at least one of the holder assemblies further configured to apply a tension to the mandrel comprises an electric motor.

In some embodiments, the system can additionally comprise a curing element arranged to move along the length of the mandrel and cure the applied layer. The curing element can comprise at least one of an air tube, a heater, an RF emitter, a UV light emitter, and an IR light emitter.

In some embodiments, the system can additionally comprise a control system programmed to regulate the operation of the one or more nozzles. In some embodiments, the system can additionally comprise a control system programmed to regulate the operation of the one or more nozzles and of the curing element. In some embodiments, the control system can be additionally programmed to regulate loading of the mandrel on or through the holding assemblies and/or to regulate cutting the mandrel after the applying. In some embodiments, the control system can be programmable to regulate a thickness of an applied and cured jacket layer to a minimum thickness of at least 0.5 microns and a maximum thickness of at most 10 microns.

A method is disclosed, according to embodiments, for manufacturing a polymeric tube. The method comprises: (a) providing an elongated mandrel between two holder assemblies; (b) sequentially depositing a plurality, or each of 5 or more, or each of 10 or more, polymeric layers over the mandrel to form an array of polymeric layers; and (c) removing the mandrel to create an elongated lumen surrounded by the array.

In some embodiments, the method can additionally comprise curing a polymeric layer. The curing can include at least one of drying, heating, exposing to a selected radio frequency (RF), and irradiating with UV or IR light.

In some embodiments, each of the sequentially applied polymeric layers can have a minimum thickness of at least 0.5 microns and a maximum thickness of at most 10 microns when cured. In some embodiments, the sequentially applying can comprise, for at least one of the polymeric layers, forming two adjoining longitudinal sections characterized by different respective hardness values. In some embodiments, the sequentially applying can comprise, for at least one of the polymeric layers, forming two adjoining longitudinal sections comprising different respective polymeric-layer material formulations. In some embodiments, the sequentially applying can comprise sequentially applying each of 25 or more polymeric layers. In some embodiments, the sequentially applying can comprise sequentially applying each of 50 or more polymeric layers.

In some embodiments of the method, an outer surface of the mandrel can be characterized by an average surface roughness of 15 to 45 microns. In some embodiments, the mandrel can comprise one (or more) of: silver-coated copper, silver-coated aluminum, and PTFE reinforced with glass fibers. In some embodiments, the mandrel can have a ductility of at least 25%.

In some embodiments, it can be that each of the sequentially applied polymeric layers has a minimum thickness of at least 0.5 microns and a maximum thickness of at most 10 microns. In some embodiments, it can be that the manufactured polymeric tube has an average surface roughness of at least 0.1 microns and not more than 5 microns.

In some embodiments, a polymeric tube can be produced by any of the foregoing embodiments. The produced polymeric tube can comprise an array of at least 10 polymeric layers each having a minimum thickness of at least 0.5 microns and a maximum thickness of at most 10 microns.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure is not limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

1. A catheter shaft produced using polymer deposition, the catheter shaft comprising:
a. a liner layer surrounding an elongated lumen;
b. a reinforcing layer at least partly surrounding the liner layer and arranged coaxially therewith, the reinforcing layer comprising a metal or metal alloy; and
c. a jacketing array at least partly surrounding the reinforcing layer and arranged coaxially therewith, the jacketing array comprising a plurality of deposited polymeric jacket layers each having a minimum thickness of at least 1 micron and a maximum thickness of at most 5 microns,
wherein at least one of the following is true:
i. the catheter shaft comprises first and second longitudinal sections characterized by different respective hardness values and separated by a transition section having a length of at least 5 mm and not more than 30 mm, the transition section being characterized by a gradation of hardness values between the different respective hardness values, and
ii. the catheter shaft comprises first and second longitudinal sections comprising different respective jacket-layer material compositions and separated by a transition section having a length of at least 5 mm and not more than 30 mm, the transition section comprising gradated intermediate blends of the different jacket-layer material formulations.

2. The catheter shaft of claim 1, wherein the catheter shaft comprises first and second longitudinal sections separated by a transition section having a length of at least 5 mm and not more than 30 mm, the first and second longitudinal sections comprising different respective jacket-layer material compositions each characterized by a different respective hardness value, wherein the transition section comprises gradated intermediate blends of the different jacket-layer material formulations and is characterized by a gradation of hardness values between the different respective hardness values.

3. The catheter shaft of claim 1, wherein the jacketing array comprises at least 5 jacket layers each having a minimum thickness of at least 1 micron and a maximum thickness of at most 5 microns.

4. The catheter shaft of claim 1, wherein the jacketing array comprises at least 10 jacket layers each having a minimum thickness of at least 1 micron and a maximum thickness of at most 5 microns.

5. The catheter shaft of claim 1, wherein the liner layer has a higher hardness value than at least one of the jacket layers.

6. The catheter shaft of claim 1, wherein the liner layer has a higher hardness value than any of the jacket layers.

7. The catheter shaft of claim 1, wherein a ratio of a maximum jacket-layer thickness to a diameter of the catheter shaft is not more than 0.2%.

8. The catheter shaft of claim 1, wherein a lengthwise majority is characterized by a monotonically increasing or decreasing inner diameter.

9. The catheter shaft of claim 1, wherein a lengthwise majority is characterized by a monotonically increasing or decreasing outer diameter.

10-22. (canceled)

23. A system for producing a catheter shaft, the system comprising:
a. a plurality of spaced-apart holder assemblies configured to support an elongated mandrel therebetween, at least one of the holder assemblies further configured to apply a tension to the mandrel; and
b. one or more nozzles, arranged to move along a length of the mandrel and deposit a jacket layer of a jacketing array while moving, the nozzles configurable to form jacket layers each having a minimum thickness of at least 1 micron and a maximum thickness of at most 5 microns, additionally comprising a control system programmed to regulate the operation of the one or more nozzles to:
i. form a jacket layer comprising first and second longitudinal sections characterized by different respective hardness values and separated by a transition section having a length of at least 5 mm and not more than 30 mm, wherein the transition section is characterized by a gradation of hardness values between the different respective hardness values,
ii. form a jacket layer comprising first and second longitudinal sections comprising different respective jacket laver material compositions and separated by a transition section having a length of at least 5 mm and not more than 30 mm, the transition section comprising gradated intermediate blends of the different jacket-laver material formulations, and/or
iii. form a jacket layer comprising first and second longitudinal sections separated by a transition section having a length of at least 5 mm and not more than 30 mm, the first and second longitudinal sections comprising different respective jacket-layer material compositions each characterized by a different respective hardness value, wherein the transition section comprises gradated intermediate blends of the different jacket-layer material formulations and is characterized by a gradation of hardness values between the different respective hardness values.

24. The system of claim 23, additionally comprising a curing element arranged to move along the length of the mandrel and cure the deposited layer while moving, the curing element comprising at least one of an air tube, a heater, an RF emitter, a UV light emitter, and an IR light emitter.

25. The system of claim 23, wherein an outer surface of the mandrel is characterized by an average surface roughness of 15 to 45 microns.

26. The system of claim 23, wherein the mandrel comprises one of: silver-coated copper, silver-coated aluminum, and PTFE reinforced with glass fibers.

27. The system of claim 23, wherein the mandrel has a ductility of at least 25%.

28-33. (canceled)
